(19) United States
(12) Patent Application Publication
(10) Pub. No.: US 2024/0293625 A1
(43) Pub. Date: Sep. 5, 2024

(54) Data Logging Device for Monitoring Use of an Injection Device

(71) Applicant: Sanofi, PARIS (FR)

(72) Inventors: William Geoffrey Arthur Marsh, Warwick (GB); Matthew Meredith Jones, Warwick (GB)

(21) Appl. No.: 18/281,222

(22) PCT Filed: Mar. 10, 2022
(86) PCT No.: PCT/EP2022/056109

§ 371 Date: Sep. 8, 2023

(30) Foreign Application Priority Data
Mar. 12, 2021 (EP) 21315034.5

(51) Int. Cl. A61M 5/315

(52) U.S. Cl. CPC A61M 5/31568; A61M 5/31555; A61M 5/3157; A61M 2205/50

(57) ABSTRACT

The present disclosure relates to a data logging device for monitoring an injection device, the injection device including a housing and a movable part movable between a first end position and a second end position, the data logging device including a trigger connectable to at least one of the movable part and the housing, a switch arrangement including a first switch engaged with the trigger and movable between a first trigger end position and a second trigger end position, the switch arrangement operable to switch between a first switching state and a second switching state upon movement of the trigger, and a processor connected to the switch arrangement and operable to detect a transition of the switch arrangement between the first switching state and the second switching state.

### CROSS REFERENCE TO RELATED APPLICATIONS

The present application is the national stage entry of International Patent Application No. PCT/EP2022/056109, filed on Mar. 10, 2022, and claims priority to Application No. EP 21315034.5, filed on Mar. 12, 2021, the disclosures of which are incorporated herein by reference.

### TECHNICAL FIELD

The present disclosure relates to a data logging device for monitoring use of a drug delivery device. In a further aspect, the disclosure relates to an injection device equipped with a data logging device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

In order to control and to supervise medication conducted by users or patients themselves it is desirable to provide an automated detecting and logging of a repeated and regular use of the drug delivery device. Especially for fixed dose injection devices typically offering a simple and rather effective approach for delivery of many drug therapies, recording of doses injected by a user would offer a significant advantage over a manual dose logging in terms of security and convenience.

### SUMMARY

Certain aspects of this disclosure relate to an improved data logging device for monitoring use of an injection device that is simple and easy to use, which is easily integratable into or adaptable to existing drug delivery devices or injection devices. The data logging device may require only a minimum amount of installation space and may be producible at low or moderate costs.

In one aspect there is provided a data logging device for monitoring use of an injection device. The injection device includes a housing and a movable part. The movable part is movable relative to the housing between a first end position and a second end position along a longitudinal direction. Typically, the housing of the injection device is an elongated housing. The injection device may be implemented as a handheld mechanically implemented injection device. The injection device may include a pen-type injection device. The movable part of the injection device may include or may constitute a dose button being manually depressible by a user of the injection device to trigger and/or to control a dispensing action of the injection device.

The data logging device is configured for monitoring use of the injection device. The data logging device may be either integrated into the injection device or may be provided as a separate device that can be fixed, e.g., releasably fixed to the injection device.

The data logging device includes a trigger connected to the movable part or being connectable to the movable part of the injection device. Alternatively, the trigger may be also connected or integrated into the housing. The trigger is movable between a first trigger end position and a second trigger end position relative to a switch arrangement.

The data logging device further includes a switch arrangement. The switch arrangement includes a first switch operably engaged with the trigger. The switch arrangement is operable to switch between a first switching state and a second switching state upon movement of the trigger relative to the switch arrangement. The switch arrangement is configured to switch into the first switching state when the trigger approaches or reaches the first trigger end position relative to the switch arrangement.

The switch arrangement is configured to switch into the second switching state when the trigger approaches or reaches the second trigger end position relative to the switch arrangement.

Typically, and with some examples the trigger is fixable to the movable part of the injection device. With other examples the trigger may be integrated into the movable part of the injection device. With some examples, the movable part and the trigger are movable in unison as the movable part is displaced by a user of the device. The trigger may be forcibly actuated or moved by the movable part. Hence, a movement of the movable part equally and unalterably transfers into a respective movement of the trigger.

With some examples the trigger is fixed to or integrated into the housing of the injection device. Then, it is the switch arrangement that is movable in unison with the movable part. Here, the switch arrangement is fixed or fixable to the movable part. As the movable part is subject to a movement relative to the housing the switch arrangement is subject to a respective movement relative to the trigger. Movement of the movable part into the first end position comes along with a respective movement of the switch arrangement to arrive in the first trigger end position relative to the trigger. Movement of the movable part into the second end position comes along with a respective movement of the switch arrangement relative to the trigger to arrive in the second trigger end position. Hence, the first and second trigger end positions always define a relative position between the trigger and the switch arrangement.

The data logging device further includes a processor. The processor is connected to the switch arrangement. The processor is operable to detect a transition of the switch arrangement between the first switching state and the second switching state. In particular, the processor is operable to detect a switching of the switch arrangement from the first switching state into the second switching state. Moreover, the processor may be operable to detect a switching of the switch arrangement from the second switching state into the first switching state.

With the present example the processor connected to the switch arrangement is operable to distinguish between two different states, configurations or positions of the trigger and hence of the movable part of the injection device. The processor is operable to determine if the switch arrangement is in the first or in the second switching state. The change of the switching state is typically only obtained when the trigger reaches or approaches one of the first or second trigger end positions that correlates with a respective first or second end position of the movable part of the injection device.

In this way and with a single switch arrangement, the data logging device will be enabled to determine if the movable part is or has been in the first end position and/or if the movable part is or has been in the second end position.

When connected to or integrated into a fixed dose injection device, wherein the injection device only includes one movable part for both setting and dispensing of a fixed dose, the data logging device will provide sufficient functionality to record setting and dispensing of a respective dose. The connection and/or mutual interaction of the processor, the switch arrangement and the trigger is sufficient to determine if the injection device is in a cocked state, i.e., in a state, in which the device is ready for dispensing of the dose, and an initial state right after conducting of a dose dispensing procedure. In some implementations, the first end position of the movable part and hence the first trigger end position relate or correspond to a cocked state of the injection device. The second end position of the movable part and hence the second trigger end position correlates with or corresponds to an initial configuration of the injection device, into which the movable part returns during a dose injection procedure.

In some implementations, the processor is operable to detect switching of the switch arrangement from the initial state, hence from the second switching state into the first switching state, a state which is typically reached at the end or during a dose setting operation. Moreover, the processor and the switch arrangement are operable to detect a subsequent return of the trigger and hence of the movable part into the second end position or trigger end position, thus indicating that a dose dispensing or dose injection procedure has been conducted and has terminated.

With some examples the first end position is a longitudinal proximal end position of the movable part. Correspondingly, the first trigger end position is a proximal longitudinal end position of the trigger. The movable part and the trigger assume this particular position relative to the housing when a dose is set or when the injection device has been cocked and is hence ready for dispensing or injecting of the dose of the medicament.

The second end position and hence the second trigger end position may be a distal end position of the movable part and hence of the trigger, which position is approached or reached at the end of a dose dispensing procedure. Hence, when a complete dose of the medicament has been injected the movable part returns into the second, e.g., in the distal end position. Likewise, the trigger reaches the respective distal second trigger end position when a dose injection procedure has terminated. Then, the injection device may be in an initial configuration.

Another dose can be set by moving the movable part from the second end position towards the first end position, thereby also moving or forcing the trigger from the second trigger end position towards and into the first trigger end position.

In the present context, the term “operably engaged” may express an engagement between a first component and a second component, such that a movement of the first component induces or causes a movement or transition of the second component.

With some examples the first switch is mechanically engaged with the trigger.

With some examples, movement of the trigger, e.g., from the first trigger end position towards and into the second trigger end position causes or induces a respective movement or switching of the first switch from the first switching state into the second switching state. Likewise, a movement of the trigger from the second trigger end position towards and into the first trigger end position may cause or induce a respective movement or switching of the first switch from the second switching state into the first switching state.

According to a further example the switch arrangement is in the first switching state when the trigger is in the first trigger end position. Moreover, the switch arrangement remains in the first switching state until the trigger approaches or reaches the second trigger end position. In this way it can be provided that the switch arrangement remains in the first switching state as long as the trigger has not yet reached the second trigger end position.

It is only upon and when reaching, or approaching the second trigger end position that the switch arrangement changes its switching configuration from the first switching state to the second switching state. The trigger reaches the second trigger end position only when the movable part reaches the second end position. Typically, the trigger and the movable part are mechanically linked or mechanically fixed to each other, such that a movement of the movable part of the injection device equally and/or unalterably transfers to a respective movement of the trigger.

Keeping the switch arrangement in the first switching state during the movement of the trigger from the first trigger end position towards the second trigger end position is beneficial to prevent a premature switching of the switch arrangement. In this way it is provided that the second switching state is only and exclusively obtained when the trigger and hence the movable part reach the second trigger end position or end position, respectively.

According to a further example, the switch arrangement is in the second switching state when the trigger is in the second trigger end position. The switch arrangement further remains in the second switching state until the trigger approaches or reaches the first trigger end position. In this way it can be provided, that the switch arrangement remains in the second switching state during a movement of the trigger from the second trigger end position towards the first trigger end position. It is only upon reaching or approaching the first trigger end position that the switch arrangement is switched from the first switching state into the second switching state. In this way, a premature switching of the switch arrangement can be effectively prevented.

The switching state is only and exclusively modified from the first switching state towards and into the second switching state when the trigger has reached the second trigger end position, which correlates with the movable part reaching the second end position.

According to another example, the processor is operable to selectively enter into one of a sleep mode and a wake-up mode. The processor is transferable from the sleep mode into the wake-up mode by switching the switch arrangement into the first switch state. This can be obtained by moving the movable part of the injection device from the second position into the first position. Accordingly, the trigger connected to the movable part may be also moved from the second trigger end position towards and into the first trigger end position. The reaching of the first trigger end position may close an electronic circuit and may provide electric energy to the processor, such that the processor enters from the sleep mode into the wake-up mode.

With a fixed dose injection device there is no necessity to measure or to detect the size of a dose actually set by a user. It is sufficient, when the data logging device and hence its processor is set into a wake-up mode during and upon a dose setting operation of the injection device. In this way and as long as the injection device is not in use, the data logging device and hence its processor may be and remain in the sleep mode. When transferred into the wake-up mode, e.g., by switching the switch arrangement from the second switching state into the first switching state, the processor and the switch arrangement are immediately ready to record a subsequent change of the switching state that occurs after or upon returning the movable part from the first end position towards and into the second end position, e.g., by pushing the movable part in distal direction.

With some examples, the processor may be configured to automatically enter into the sleep mode after lapse of a predetermined time interval after completion of a dose dispensing procedure, i.e., after the switch arrangement has been switched into the second switching state.

With some examples, the processor may automatically switch into the sleep mode when the switch arrangement is in the second switching state and when no further change of the switching state occurs during a predefined time interval. Such an automated activation of the sleep mode may be beneficial to save electrical energy and to prolong lifetime of the data logging device.

According to a further example, the processor is operable to log and/or to record a dose dispensing event when detecting a switching of the switch arrangement from the first switch state into the second switch state. Typically, each time the switch arrangement is switched from the first switching state into the second switching state such a detection is evaluated as completion of a dose dispensing or dose injection procedure.

The processor can be equipped with or connected to an electronic memory, and is then operable to store dose dispensing related data in the electronic memory. Additionally or alternatively, the processor may be operable to transmit a dose completion signal to an external data logging device, such as an external electronic device. Then, the functionality of the processor may be further limited. It may be sufficient, when the processor is exclusively operable to monitor the change of the switching state of the switch arrangement and to generate a switching state changing signal every time when the switch arrangement changes its switching state. This way the hardware for the data logging device can be simplified and offers a reduction of costs for manufacturing of the data logging device.

With further examples the data logging device includes an electronic memory operable to store or to log a dose dispensing event conducted by the injection device. Typically, the electronic memory is operable to store dispensing related data, such as a date or point of time at which the injection took place. Optionally, the electronic memory may also store a particular size of a dose that has been injected. With a fixed dose injection device, the size of the dose is not subject to modifications and remains constant for each subsequent dose dispensing procedure. Insofar, the data to be stored in the electronic memory may be limited to a time or date information at which a dose of fixed and hence known size has been actually dispensed or injected.

In some implementations, the data logging device also includes an energy reservoir. The energy reservoir typically includes an electric battery. The battery may include a button battery of rather compact size that allows to minimize the construction space for the data logging device.

According to another example, the data logging device includes a communication interface to transmit dose dispensing related data to an external electronic device. The communication interface is connected to the processor. Here, the processor may be operable to read-out data from the electronic memory and to transmit the data via the communication interface to the external electronic device.

The communication interface may be implemented as a wireless communication interface operable to establish a wireless data connection to the external electronic device. In some implementations, the wireless communication interface is configured to set up a wireless communication link to the external electronic device. Here, different wireless communication standards, such as RF-based communication standard can be used, such as Wi-Fi, NFC or Bluetooth®.

According to a further example of the data logging device, at least one of the trigger and the switch arrangement provide a hysteresis function. Alternatively, the hysteresis function is provided by the mutual operable engagement of the trigger and the switch arrangement. The hysteresis function is operable to prevent a change of the switching state of the switch arrangement as long as the trigger is located between the first trigger end position and the second trigger end position relative to the switch arrangement. By way of the hysteresis function it can be provided, that a change of the switching state only and exclusively takes place when the trigger is in or reaches the first end position or the second end position relative to the switch arrangement.

In this way and when for instance moving the trigger or the movable part of the injection device from the first end position only half way towards the second end position the switch arrangement remains in the first switching state. The other way round and when the switch arrangement is in the second switching state the hysteresis function is operable to prevent a premature switching of the switch arrangement into the first switching state, e.g., when the movable part or the trigger are subject to a movement from the first end position only half way towards the second end position but not reaching the second end position.

The hysteresis function helps to prevent a premature change of the switching state before the movable part or the trigger has reached a respective end position.

By way of the hysteresis function it should be guaranteed, that a change of the switching state only occurs when the movable part of the injection device and hence when the trigger of the data logging device has reached one of the first or second end position.

According to a further example, the hysteresis function is electronically implemented in the switch arrangement. Here, the first switch of the switch arrangement may be subject to a gradual and continuous switching movement as the trigger is moved from the first trigger end position towards the second trigger end position; or vice versa. The gradual movement of the first switch may be recorded and analyzed by integrated electronics of the switch, the switch arrangement or of the processor. A gradual movement of, e.g., a peg of a mechanical switch may be in close correspondence to the respective gradual movement of the trigger being subject to a movement between the first and second trigger end positions. Here, the integrated electronics of the switch, of the switch arrangement or of the processor may be electronically detected to determine when the first or second trigger end positions and hence a respective switch end position has been reached. A respective switching may then only occur upon detection of such an end position.

According to another example the hysteresis function is mechanically implemented in one of the switch arrangement and the trigger. Alternatively, the hysteresis function is mechanically implemented by the operable engagement of the trigger and the switch arrangement. A mechanical implementation of the hysteresis function may be obtained in numerous different ways.

With a further example, the hysteresis function may be implemented by introducing an asymmetry in the trigger by way of which the respective switching points of the first switch are shifted such that the first switch enters into the first switch state, e.g., an open or closed state when the trigger reaches the first trigger end position. The first switch may enter into the second switching state only when the trigger reaches or is very close to the second trigger end position.

According to another example, the first switch is a mechanical switch. The mechanical switch includes a switch housing and a peg. The peg protrudes from the switch housing and is movable relative to the switch housing against the action of a return spring. The return spring may be supported by the housing. The return spring may be arranged inside the housing. The spring is further mechanically connected to the peg. In this way, the peg may be depressed into the housing against the action of the return spring. Release of the peg may lead to a spring-driven return motion of the peg and a respective movement of the peg outwardly relative to the switch housing.

The first switch may be implemented as a micromechanical switch. The switch, for example its peg, may be in abutment or in sliding engagement with the trigger. Here, a movement of the trigger relative to the first switch may lead to a depression of the peg so as to close or to open an electric circuit interrupted by the mechanical which.

According to a further example the peg is movable along a transverse direction relative to the switch housing. The trigger or at least a portion thereof is movable, pivotable, or bendable in the transverse direction when the trigger moves between the first trigger end position and the second trigger end position. For example, the trigger may be displaceable relative to the switch arrangement along the longitudinal direction. The trigger may simply follow the movable part of the injection device. With some examples, the trigger itself may be implemented as a follower connected or fixed to the movable part of the injection device.

The trigger and the movable part may be movable relative to the housing of the injection device in longitudinal direction during a movement of the movable part between the first end position and the second end position. The trigger may be further movable in a transverse direction, e.g., perpendicular to the longitudinal direction in order to activate or to deactivate the first switch of the switch arrangement. With some examples, the peg of the mechanical switch may point or protrude in a transverse, e.g., a radial direction, whereas the trigger or the movable part is movable along the longitudinal direction. As the trigger and/or the movable part is moved in longitudinal direction relative to the mechanical switch it may become subject to a movement, pivoting or bending motion in the transverse direction.

The trigger may be configured to redirect a longitudinal movement of the movable part into a radial or transverse movement of the peg of the switch. Here, a kind of a mechanical transmission can be implemented by way of which the displacement path of the movable part and hence of the trigger can be adapted to the displacement path of the peg of the mechanical switch.

According to a further example, the trigger includes a trigger body movable relative to the housing along the longitudinal direction. The trigger body includes a beveled section, which, when engaged with the housing being operable to induce at least one of a movement, a pivoting or a bending of the trigger or at least a portion thereof in the transverse direction. The beveled section is typically provided at a longitudinal end of the trigger. It may be provided at a longitudinal portion of the trigger that substantially coincides with the longitudinal position of the mechanical switch. The beveled section may be provided on a first surface section of the trigger and the trigger may include an oppositely located second surface section configured to engage or to abut with the mechanical switch, in particular with the peg of the mechanical switch.

In this way and as the trigger is subject to a longitudinal movement relative to the housing and as the beveled section engages, e.g., a complementary shaped section of the housing, the trigger, e.g., its beveled section, may become subject to a movement, pivoting or bending in transverse direction. The surface section of the trigger facing towards the mechanical switch may move further towards the mechanical switch and may thus press the peg to change the switching state of the mechanical switch.

The implementation of a beveled section of the trigger body is rather simple and provides a well-defined and easily controllable depression of the peg of the mechanical switch when the trigger is subject to a longitudinal movement relative to the housing of the injection device.

According to another example, the trigger includes a slotted link in which the peg of the mechanical switch is guided. The slotted link may include a closed loop. The slotted link includes a first longitudinal end section and a second longitudinal end section. A transverse depth of the slotted link in the first longitudinal end section distinguishes from a transverse depth of the slotted link in the second longitudinal end section. In this way, a switching movement of the peg in the transverse direction can be induced as the peg slides from, e.g., the first longitudinal end section with the first transverse depth into the second longitudinal end section featuring the second transverse depth.

The bottom of the slotted link or a sidewall of the slotted link may include a respective beveled section or a ramp section by way of which the first transverse depth provided in the first longitudinal end section smoothly merges or transitions into the second transverse depth as provided in the second longitudinal end section. Respective transition areas, e.g., in form of ramped sections, are typically provided near or at the respective first and second longitudinal end sections of the slotted link in order to provide the above-mentioned hysteresis function.

Hence, the slotted link of the trigger in which the peg is slidably guided is another example of a mechanically implemented hysteresis function. Here, the trigger may be slightly rotatable or bendable in circumferential direction, i.e., perpendicular to the longitudinal and perpendicular to the transverse direction so as to enable a smooth sliding of the peg of the mechanical switch in the slotted link as the trigger is subject to a movement from the first trigger end position towards and into the second trigger end position along a first path of the slotted link and as the trigger is subject to a return movement from the second trigger end position towards and into the first trigger end position along a second path of the slotted link. Typically, first and second paths of the slotted link do not spatially overlap.

According to a further example, the switch arrangement includes a first switch and a second switch. The first switch and the second switch are each connected to the processor. The first switch and the second switch are both operably engaged with the trigger. The first switch is operable to detect approaching or arriving of the trigger in the first trigger end position. The second switch is operable to detect approaching or arriving of the trigger in the second trigger end position. Both switches provide an individual or a separate signal to the processor. Based on a first signal obtained from the first switch and based on a second signal obtained from the second which the processor may then distinguish between first and second switching state of the switch arrangement, wherein the switching state of the switch arrangement characterizes the momentary configuration or position of the movable part of the injection device.

Accordingly, and with a first and a second switch, the first and the second trigger end positions can be individually and precisely detected. For instance, the first switch only closes when the trigger reaches the first trigger end position relative to the switch arrangement. Correspondingly, the second switch only and exclusively closes when the trigger reaches the second trigger end position relative to the switch arrangement. In the course of moving the trigger, e.g., from the first trigger end position towards the second trigger end position the first switch may open before the second switch will be closed.

Such a configuration may be regarded and evaluated by the processor as an invalid switching state. Here, both switches may exhibit the same individual switch state. When the trigger reaches the second trigger end position the second switch may be closed while the first switch may be opened. Such a heterogeneous configuration of the first and the second switches can be evaluated as a valid switching state of the switch arrangement.

For example, the second switching state of the switch arrangement may be characterized by the second switch being in a closed configuration and the first switch being in an open configuration. The first switching state of the switch arrangement may be defined by the first switch being closed and the second switch being open.

With other examples, an inverted switch behavior may be implemented, wherein the first switching state of the switch arrangement is obtained when the second switch is closed and the first switch is opened. The second switching state of the switch arrangement may be obtained when the second switch is open and when the first switch is closed.

When the first and the second switch of the switch arrangement are in the same binary switch state, e.g., when both switches are open or when both switches are closed this may be regarded or evaluated as an invalid switching state of the switch arrangement. Here, the trigger position is located between the first trigger end position and the second trigger end position and should be disregarded for the data logging. Implementation of a first switch and a second switch of the switch arrangement may provide an electronic or electromechanical approach for implementing a hysteresis function.

According to a further example, the first switch or the second switch includes one of an electromechanical switch, a magnetic switch, an optical sensor, or an acoustic sensor. When implemented as an optical sensor, the switch may include a photodiode or a comparable light-sensitive element. When implemented as an acoustic sensor, the respective switch may include a microphone being sensitive to a well-defined noise or click sound produced by the trigger when reaching one of the first and second trigger end positions.

With some examples, the first switch and the second switch are implemented in the same way. Hence, the first switch and the second switch are implemented as an electromechanical switch, as a magnetic switch, as an optical sensor, or as an acoustic sensor. With other examples, one of the first switch and the second switch is implemented as an electromechanical switch, a magnetic switch, an optical sensor, or an acoustic sensor whereas the other one of the first switch and the second switch is implemented as another one of the electromechanical switch, a magnetic switch, an optical sensor, or an acoustic sensor.

In this way different switch principles and technical implementations of switches can be realized thus allowing to reduce construction space of the data logging device or to increase precision of the trigger end position detection.

According to a further example, the data logging device includes a logging device housing configured for attachment to the housing of the injection device. Here, the trigger is configured for attachment to the movable part of the injection device. In some examples, the injection device includes a device fastening feature and the data logging device includes a logging device fastening feature complementary shaped to the device fastening feature. In this way the data logging device can be releasably or detachably connected and fixed to the housing of the injection device. At the same time, also the trigger may include a fastening feature complementary shaped to a corresponding counter fastening feature of the movable part of the injection device. Fastening features and respective counter fastening features of the data logging device and of the injection device may be shaped and configured to establish at least one of a form fitting engagement and/or friction fitting engagement.

With another example, the logging device housing is configured for attachment to the movable part of the injection device. Here, the trigger may be configured to engage with the housing of the injection device. Also with this example there may be provided respective fastening features of the data logging device and of the injection device to provide a mutual and releasable fastening thereof.

By enabling a releasable attachment of the data logging device to the injection device existing injection devices can be retrofitted with a data logging device. The releasable or detachable fixing of the data logging device to the injection device further allows to use the data logging device with a plurality of, e.g., disposable injection devices. Once the injection device has reached an end of content configuration and should be discarded the data logging device may be detached from the injection device and can be attached to a new injection device. In this way, the data logging device including electronic components and a battery can be used with numerous disposable injection devices.

The releasable attachment of the data logging device with the injection device enables a rather long lifetime for the data logging device and provides a data monitoring and data logging over a comparatively long time interval.

According to another aspect, the disclosure relates to an injection device for injecting of a dose of a medicament into biological tissue. The injection device includes a housing to accommodate a cartridge filled with an injectable medicament. The cartridge is sealed towards a longitudinal proximal direction by a piston. The piston is movable relative to a barrel of the cartridge. The injection device includes a piston rod to operably engage with the piston in order to expel a dose of the medicament from the cartridge. Typically, the medicament is expelled by moving the piston rod in distal direction, thereby urging the piston of the cartridge to move in distal direction relative to the barrel of the cartridge.

The injection device further includes a movable part, e.g., in form of a dose button. The movable part is movable relative to the housing of the injection device between a first end position and a second end position along a longitudinal direction. The injection device further includes a data logging device as described above. Here, the data logging device, e.g., the trigger, the switch arrangement and the processor of the data logging device as described above are assembled and arranged inside or on the injection device.

In one aspect of the disclosure, the data logging device is integrated into the injection device. With other aspects, the data logging device is provided as a separate and stand-alone device configured for coupling with the injection device.

The movable part of the injection device may be mechanically engaged with the piston rod. The injection device may include a drive mechanism providing a mechanical link between the movable part and the piston rod. A user exerting a dispensing force onto the movable part, e.g., depressing the movable part in distal direction relative to the housing of the injection device, may be transferred mechanically to the piston rod, such that the piston rod advances in distal direction for expelling of the dose of the medicament. Insofar and with some examples the drive mechanism of the injection device provides a mechanical transmission to transfer the expelling force applied by a user onto the movable part into an advancing force applied by the piston rod onto the piston in distal direction for dose expelling.

In some implementations, the injection device is implemented as a fixed dose injection device. Here, the user is only given the possibility to set a dose or to cock the injection device and to subsequently trigger or to initiate a respective dose expelling procedure. The movable part of the injection device may be in slidable engagement with the drive mechanism and/or with the housing. It may be movable back and forth in longitudinal direction relative to the housing for setting of a dose and for injecting of the dose.

According to a further example the data logging device is arranged inside at least one of the housing of the injection device and the movable part. Here, the trigger is connected to the movable part. The trigger may be fixed to the movable part, such that any longitudinally directed movement of the movable part is equally and unalterably transferred into a respective longitudinal, e.g., sliding movement of the trigger relative to the housing and/or relative to the switch arrangement of the data logging device.

According to a further example the trigger of the data logging device is integrated into the movable part. With further examples the trigger includes a resilient arm to slidably engage with a sidewall of the housing of the injection device when the movable part is moved between the first end position and the second end position. The resilient arm may be flexible or pivotable in a transverse direction, e.g., perpendicular to the longitudinal direction of the housing as well as perpendicular to the longitudinal sliding movement of the movable part and/or of the trigger.

The resilient arm of the trigger and/or of the movable part being, e.g., resiliently deformable, flexible or bendable, or even pivotable in transverse, e.g., in radial direction, is beneficial in combination with a mechanical switch with a peg being movable in the respective transverse, e.g., radial direction. In this way, a longitudinal displacement of the movable part and/or of the trigger can be redirected or transferred into a radial displacement. This redirection or transfer of a displacement is beneficial to adapt the total displacement path of the movable part to a maximum available displacement path of the peg of the mechanical switch.

In this way, a commercially available mechanical switch, e.g., providing a limited displacement path for its peg can be used and adapted for detecting a displacement path of a movable part between the first and the second end position, which may larger than the maximum available displacement path of the peg of the mechanical switch. Also, with the redirection of the displacement of the end position detection provided by the mechanical switch can be further improved.

According to a further example the injection device is transferable from an idle or initial state into a cocked state or dose setting state by moving the movable part from the second end position towards and into the first end position. Here, the second end position may coincide or represent the idle state. The first end position may coincide or define a cocked state or a state, in which the injection device is prepared for dispensing or injecting of the dose.

Hence, the movable part may be configured for a sliding back and forth movement between the first and the second end positions, wherein the second end position coincides with an initial or idle state of the injection device.

According to a further example of the injection device, a fixed dose of the medicament is injectable or dispensable by moving the movable part from the first end position towards and into the second end position. Here and with further examples the driving force required for moving the piston rod in distal direction is entirely provided by a user exerting a respective dispensing force onto the movable part of the injection device. Typically, the movable part of the injection device is located at a longitudinal proximal end of the injection device. The injection device may be implemented as a pen-type injector. The movable part, e.g., implemented as a dose button may be depressible by a thumb or finger of a user for injecting the dose of the medicament.

According to a further example the piston rod is movable in a longitudinal distal direction by and/or through moving the movable part from the first end position towards and into the second end position. The distance of travel of the piston rod during a dose dispensing or dose injecting procedure may be proportional to the displacement of the movable part from the first end position towards the second end position.

According to a further example a longitudinal displacement of the movable part relative to the housing from the first end position towards and into the second end position is proportionally transmitted into a respective longitudinally directed advancing displacement of the piston rod for injecting of the dose of the medicament.

Typically, the drive mechanism providing a respective transfer of driving forces from the movable part to the piston rod provides a kind of a reduction gear. In this way, the displacement path of the movable part from the first end position to the second end position is larger than the displacement of the piston rod in the distal direction during a dose dispensing procedure. With the reduction gear as provided by the drive mechanism a respective force amplification for driving the piston rod in distal direction can be provided.

According to a further example the injection device is equipped with the cartridge being filled with the injectable medicament. Here, the cartridge with the medicament is arranged inside the housing. The injection device may be implemented as a disposable injection device, wherein the cartridge with the medicament is preassembled inside the housing of the injection device. The housing of the injection device may include at least a first and a second housing part, wherein a first housing part is configured to accommodate the cartridge and wherein the second housing part is configured to accommodate the drive mechanism or at least the piston rod.

With a disposable injection device the first and the second housing parts may be non-releasably or non-detachably connected. Here, detachment of first and second housing part is only possible by destroying or damaging at least one of the respective housing parts.

With other examples the injection device is implemented as a reusable injection device allowing a detachment or a relative movement of first and second housing components for replacing the cartridge. With reusable injection devices the dose setting mechanism may provide a reset function so as to restore the initial configuration of the dose setting mechanism when a new cartridge is used with the injection device.

According to another aspect the disclosure also relates to a method of data logging and/or monitoring use of an injection device. The method includes the use of a data logging device and/or of an injection device equipped with a logging device as described above. The method includes the step of moving a movable part of the injection device from a second end position towards and into a first end position. Concurrently, a trigger of the data logging device is moved from a second trigger end position into a first trigger end position. Approaching or reaching of the first trigger end position is detected by a switch arrangement.

Accordingly, the switch arrangement is switched into a first switching state. Thereafter the movable part is moved from the first end position towards and into the second end position. The trigger is moved accordingly from the first trigger end position towards and into the second trigger end position. Approaching or reaching of the second trigger end position provides switching of the switch arrangement from the first switching state into the second switching state. The change of the switching state, e.g., the change of the first switching state into the second switching state and/or the change of the second switching state into the first switching state is detected by a processor.

Upon detecting a state change of the switch arrangement the processor is either transferred into a wake-up mode and/or the processor logs or stores a dose dispensing event. Optionally or additionally, the processor transfers dispensing related data to an external electronic device, e.g., via a communication interface.

Generally, the scope of the present disclosure is defined by the content of the claims. The injection device is not limited to specific embodiments or examples but includes any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not include a full-length antibody polypeptide, but that still includes at least a portion of a full-length antibody polypeptide that is capable of binding to an antigen. Antibody fragments can include a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, numerous examples of a data logging device for monitoring use of an injection device as well as a respective injection device will be described in greater detail by making reference to the drawings, in which:

FIG. 1 shows an example of an injection device in a longitudinal cross-section,

FIG. 2 shows a perspective illustration of a proximal end of the injection device with a movable part in a first end position,

FIG. 3 shows the injection device according to FIG. 2 with the movable part in the second end position,

FIG. 4 is a schematic block diagram of a data logging device,

FIG. 5 is a schematic illustration of numerous components of the data logging device when integrated or arranged in the injection device,

FIG. 6 is a flowchart of a method of data logging,

FIG. 7 is a perspective cross-section through the injection device of FIG. 2,

FIG. 8 is a perspective cross section through the injection device according to FIG. 3,

FIG. 9 schematically shows the interaction of the housing, the trigger and the switch arrangement in the first switching state,

FIG. 10 shows the example of FIG. 9 with the trigger in the second trigger end position,

FIG. 11 schematically illustrates the switch implemented as a mechanical switch in a first switch configuration,

FIG. 12 shows the switch according to FIG. 11 in the second switch configuration,

FIG. 13 is illustrative of a diagram of a hysteresis function provided by the switch arrangement,

FIG. 14 shows a mechanical implementation of a hysteresis function,

FIG. 15 shows the transverse depth of the slotted link of FIG. 14 along first and second paths of the slotted link,

FIG. 16 shows another implementation of the trigger and the switch arrangement,

FIG. 17 shows a further implementation of the switch arrangement including a first and a second switch in a first switching state, and

FIG. 18 shows the switch arrangement of FIG. 17 in the second switching state.

### DETAILED DESCRIPTION

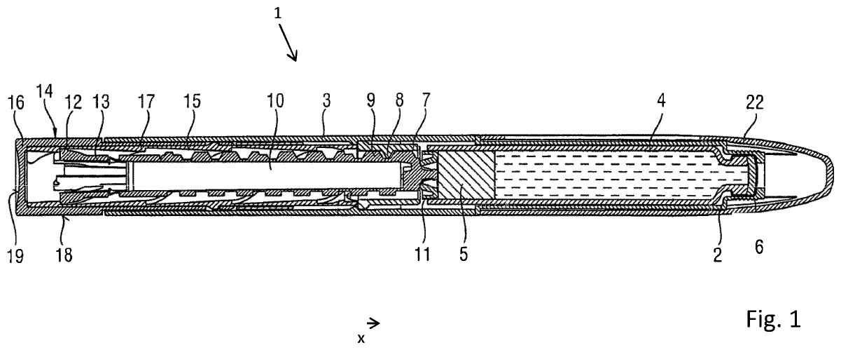

In FIG. 1, there is shown a drug delivery device implemented as a handheld injection device, e.g., an injection pen. The drug delivery device 1 includes a cartridge retaining part 2, and a main (exterior) housing part 3. The proximal end of the cartridge retaining part 2 and the distal end of the main housing 3 are secured together.

A cartridge 4 from which a number of doses of a medicinal product may be dispensed is provided in the cartridge retaining part 2. A piston 5 is retained in the proximal end of the cartridge 4. A removable cap 22 is releasably retained over the distal end of the cartridge retaining part 2. The removable cap 22 and/or the retaining part is optionally provided with one or more window apertures through which the position of the piston 5 within the cartridge 4 can be viewed.

The distal end of the cartridge retaining part 2 is provided with a distal threaded region 6 designed for the attachment of a suitable needle assembly (not shown) to enable medicament to be dispensed from the cartridge 4 and injected into biological tissue.

The main housing part 3 is provided with an internal housing 7. The internal housing 7 is secured against rotational and/or axial movement with respect to the main housing part 3. The internal housing 7 is provided with a threaded circular opening 8 extending through the distal end of the internal housing 7. The threaded circular opening 8 includes a series of part threads rather than a complete thread. Alternatively, the internal housing 7 may be formed integrally with the main housing part 3. Additionally, the internal housing 7 may be provided with a plurality of guide slots and pawl means.

A first thread 9 is formed at the distal end of the piston rod 10. The piston rod 10 is of generally circular cross-section. The first thread 9 of the piston rod 10 extends through and is threadedly engaged with the threaded circular opening 8 of the internal housing 7. A pressure foot 11 is located at the distal end of the piston rod 10. The pressure foot 11 is disposed to abut the proximal face of the piston 5. A second thread 12 is formed at the proximal end of the piston rod 10. In the illustrated embodiment the second thread 12 includes a series of part threads, rather than a complete thread, formed on flexible arms 13 of the piston rod 10.

The first thread 9 and the second thread 12 are oppositely disposed. The first thread 9 is provided with a plurality of features (not shown) that cooperate with the part threads of the threaded circular opening 8 to prevent movement of the piston rod 10 in the proximal direction during setting of the device. A drive sleeve 14 extends about the piston rod 10. The drive sleeve 14 includes a threaded part 15 of a generally cylindrical cross-section and an activation or movable part 16. The threaded part 15 and the activation part 16 are secured to each other to prevent rotational and/or axial movement there between. Alternatively, the drive sleeve 14 may be a unitary component consisting of an integrated threaded part 15 and activation part 16. The activation part may serve as a dose button manually operable by a user for setting and for dispensing of a fixed dose of the medicament. In the following, the activation part 16 may be also denoted as a movable part since it is longitudinally movable relative to the housing 3.

In the illustrated embodiment, the threaded part 15 is provided with a longitudinally extending helical thread 17 formed on the internal cylindrical surface. The flank of the distal side of the helical thread 17 is designed to maintain contact with the second thread 12 of the piston rod 10 when dispensing a dose, whilst the flank of the proximal side of the helical thread 17 is designed to allow the second thread 12 of the piston rod 10 to disengage when setting a dose. In this way the helical thread 17 of the threaded part 15 is releasably engaged with the second thread 12 of the piston rod 10.

The drive sleeve 14 has a plurality of features formed on the external surface designed to move axially within the guide slots of the internal housing 7. These guide slots define the extent of permissible axial movement of the drive sleeve 14 with respect to the housing part 3. The guide slots also prevent rotational movement of the drive sleeve 14 relative to the main housing part 3.

The activation part or movable part 16 of the drive sleeve 14 has a plurality of grip surfaces 18 and a dispensing face 19. The drive sleeve 14 is provided with a detent means that is designed to interact with the pawl means of the internal housing 7. To increase intuitiveness of the operation of the device, the main housing part 3 may be provided with a window aperture through which graphical status indicators, provided on the drive sleeve 14, can be viewed.

Operation of the drug delivery device will now be described. To set a dose a user grips the grip surfaces 18 of the drive sleeve 14, in particular, the user grips the movable part 16. The user then pulls the movable part 16 and hence the drive sleeve 14 in a proximal direction away from the main housing part 3.

The piston rod 10 is prevented from moving proximally by the part threads of the threaded circular opening 8 of the internal housing 7 interacting with thread features on the first thread 9 of the piston rod 10 or by any other suitable means. As the drive sleeve 14 travels in the proximal direction relative to the piston rod 10, the second thread 12 of the piston rod 10 is displaced radially inwards by the flank of the proximal side of helical thread 17 of the drive sleeve 14.

The proximal travel of the drive sleeve 14 is limited by the guide slots (not shown) of the internal housing 7 a distance corresponding to essentially one thread pitch of the helical thread 17 of the drive sleeve 14. At the end of the travel of the drive sleeve 14, the second thread 12 of the piston rod 10 engages with the helical thread 17 under the action of the flexible arms 13 of the piston rod 10. By this action the drive sleeve 14 is displaced a distance essentially equal to one pitch of the helical thread 17 of the drive sleeve 14 in the proximal direction relative to the piston rod 10. The action of the second thread 12 positively engaging the helical thread 17 of the drive sleeve 14 under a force provided by the flexible arms 13 creates an audible and tactile feedback to the user to indicate that the dose has been set. Additionally, visual feedback regarding dose setting may be indicated by an optional graphical status indicator, provided on the drive sleeve 14, which can be viewed through an optional window aperture in the main housing part 3.

When the dose has been set, the user may then dispense this dose by depressing the dispensing face 19 of the movable part 16. By this action the drive sleeve 14 is moved axially in the distal direction relative to the main housing part 3. As the second thread 12 of the piston rod 10 is positively engaged with the helical thread 17 of the drive sleeve 14 the piston rod 10 is caused to rotate with respect to the internal housing 7 by the axial movement of the drive sleeve 14 in the distal direction. As the piston rod 10 rotates, the first thread 9 of the piston rod 10 rotates within the threaded circular opening 8 of the internal housing 7 causing the piston rod 10 to move axially in the distal direction with respect to the internal housing 7.

The distal axial movement of the piston rod 10 causes the pressure foot 11 to bear against the piston 5 of the cartridge 4 causing a dose of medicament to be dispensed through an attached needle.

The distal travel of the drive sleeve 14 is limited by the guide slots (not shown) of the internal housing 7. Audible and tactile feedback to indicate that the dose has been dispensed is provided by the interaction of the detent (not shown) of the drive sleeve with the pawl means (not shown) of the internal housing 7. Further doses of equal size may be delivered as required up to a pre-determined maximum number of doses. The mechanical function and operation of the injection device 1 as illustrated in FIG. 1 is also described in greater detail in WO 2008/058665 A1, the entirety of which being incorporated herein by reference. The injection device and drive mechanism exercising FIG. 1 is only one example of an injection device that can be used with the data logging device 30 of the present disclosure.

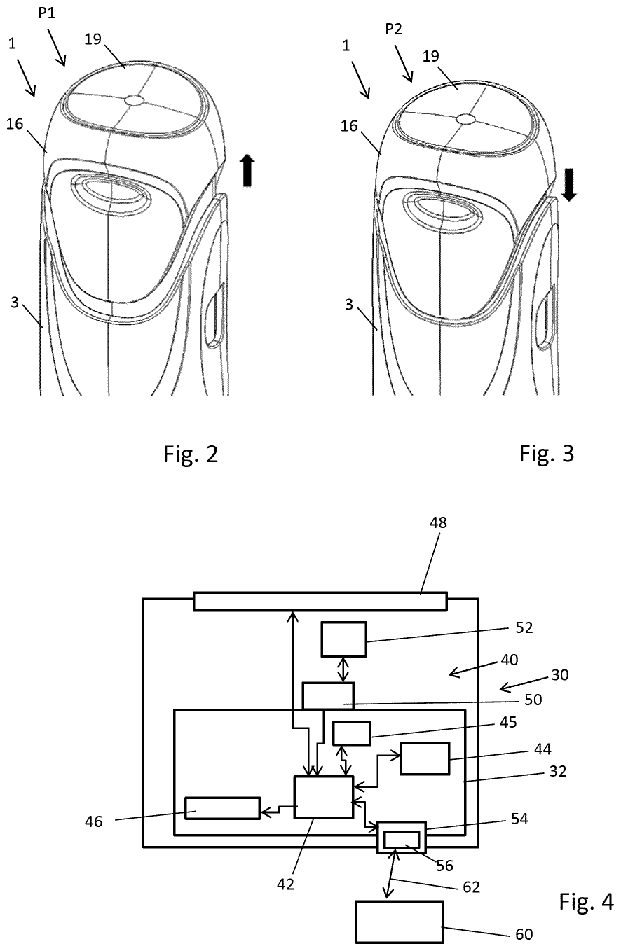

The injection device one as illustrated in FIG. 1 is particularly implemented as a fixed dose injection device. The activation part 16 or movable part 16 is movable back and forth in longitudinal direction x for setting of the dose and for subsequently dispensing or injecting of the dose. For setting of the dose and/or for transferring the injection device 1 into a cocked state the movable part 16 is to be pulled in proximal direction so that it extends in proximal direction from the housing part 3. In this configuration, the movable part 16 is in a first end position P1, which is further illustrated in FIG. 2.

For dispensing of the dose, of the medicament the movable part 16 is depressible in distal direction as illustrated in FIG. 3. During and while the movable part 16 is depressed and moved in distal direction, e.g., by a thumb of a user, the piston rod 10 is subject to a respective distally directed displacement, thereby expelling the dose of the medicament. When returning into an initial configuration the movable part 16 reaches the second end position P2.

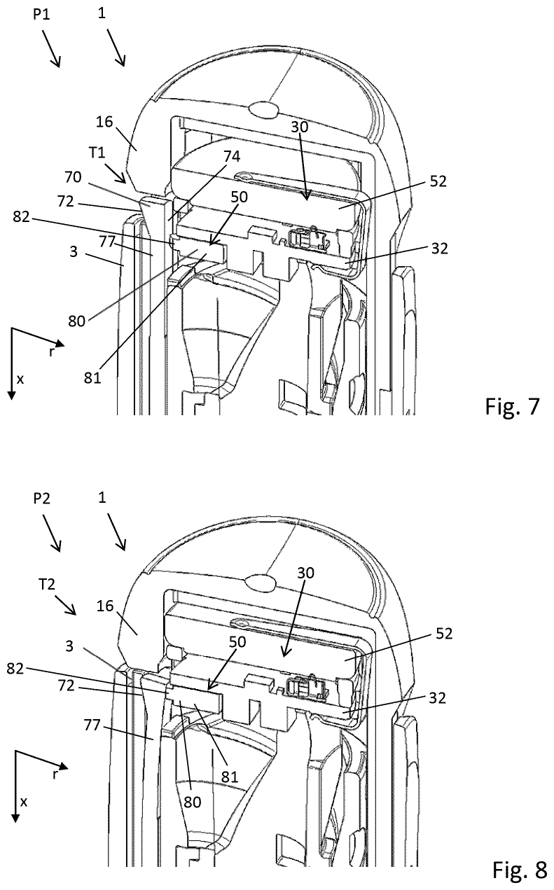

In the cross-section of FIGS. 7 and 8, the configurations of FIGS. 2 and 3 are reproduced again. With the example of FIGS. 7 and 8 a data logging device 30 is integrated and arranged inside the housing 3 as well as inside the movable part 16. The data logging device 30 includes a trigger 70 and a switch arrangement 50. The data logging device 30 further includes a processor 42 connected to the switch arrangement 50 and operable to detect a transition of the switch arrangement 50 between a first switching state i and a second switching state ii.

The switch arrangement 50 as illustrated in FIGS. 7 and 8 includes a first switch 80. The switch 80 is implemented as a mechanical switch 81. One example of such a mechanical switch is illustrated in FIGS. 11 and 12. There, the mechanical switch 81 includes a switch housing 83 and a peg 82 protruding outwardly from the switch housing 83. The peg 82 is supported by a return spring 84. The peg 82 is depressible inside the switch housing 83 against the action of the return spring 84. In a first switching state i, the peg 82 protrudes outwardly from the housing 83.

The peg 82 is depressible further into the housing to reach a second switching state ii as illustrated in FIG. 12. Here, the spring 84 is biased. Releasing of the peg 82 leads to a return motion of the peg 82 into the first switching state i as illustrated in FIG. 11.

With the specific implementation of the data logging device 30 as illustrated in FIGS. 7 and 8 the mechanical switch 81 of the switch arrangement 50 includes the peg 82 protruding in a transverse or radial direction r towards an inside facing surface section 74 of a trigger 70. The trigger 70 is implemented as a flexible arm 77. With the present example the trigger 70 is integrated into a sidewall of the movable part 16. The movable part 16 may include a cup-shaped receptacle to accommodate the data logging device 30. The switch arrangement 50 is attached or assembled to a printed circuit board 32. It is either fixed inside the movable part 16 or it is fixed inside the housing 3.

With the example of FIGS. 7-10 the switch arrangement 50 is fixed to the housing 3 of the injection device 1 whereas the movable part 16 and the trigger 70 are movable in longitudinal direction relative to the housing 3.

As illustrated in FIGS. 7-10 and when in the first end position P1 the trigger 70 is in a first trigger end position T1. The trigger 70 includes an outward facing surface section 73 which may be in a slidable engagement with an inside surface of a sidewall of the housing 3. Towards its proximal end the outside facing surface 73 of the trigger 70 includes a radially outwardly protruding beveled section 72. When in the first trigger end position T1 the beveled section 72 is located proximally offset from a respective proximal end of the housing 3. In this way, the outwardly facing surface section 73 of the trigger 70 is in abutment or in sliding engagement with an inside surface of the housing 3.

As the trigger 70 is subject to a distally directed displacement towards the second trigger end position T2 as illustrated in FIGS. 8 and 10 the beveled section 72 engages and slides along the inside of the sidewall of the housing 3, thereby inducing a radially inwardly directed movement of the proximal end section of the trigger 70. The switch arrangement 50 is longitudinally and radially fixed relative to the housing 3. The trigger 70 is located radially between the switch arrangement 50 and the housing 3. Accordingly, the radially inwardly directed deflection or bending of the trigger 70 as induced by the beveled section 72 when sliding along the sidewall of the housing 3 is operable to induce a movement of the peg 82 of the switch 80 into the second switching state ii.

Only and when the movable part 16 as well as the trigger 70 reach the second end position and hence the second trigger end position T2 the switch arrangement 50 changes from the first switching state i into the second switching state ii. This change of the switching state is detected by the processor 42.

The trigger 70 includes a trigger body 71 featuring an inside surface section 74 being, e.g., permanently engaged with the free end of the peg 82 of the mechanical switch 81. In this way and when the proximal end of the trigger body 71 is subject to a deflection radially inwardly as induced by the beveled section 72 the peg 82 is subject to a respective movement in radial direction r or a transverse direction.

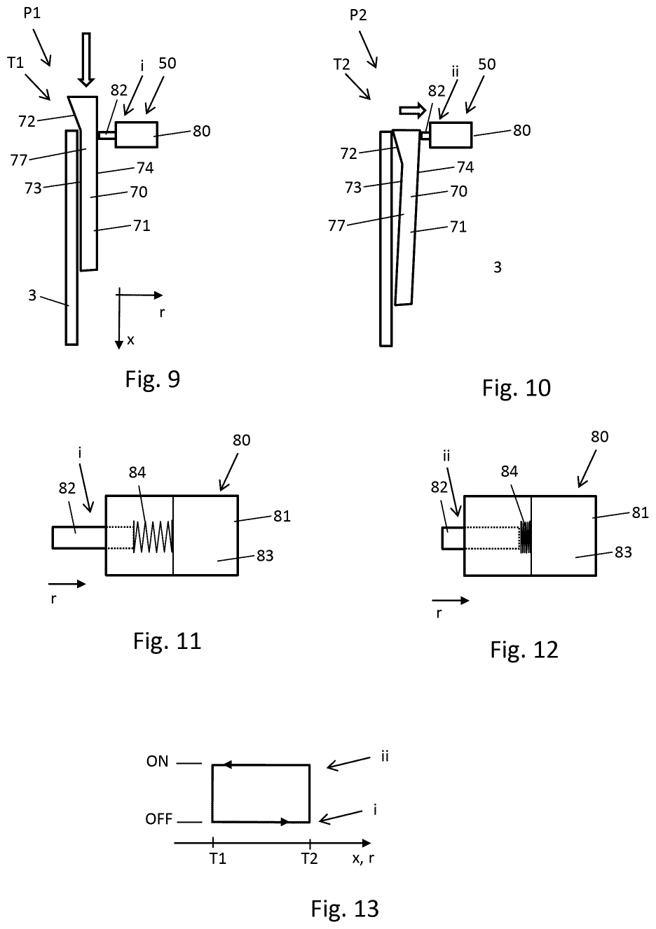

When the movable part 16 and hence the trigger 70 move from the second end position P2 towards and into the first end position P1 the trigger will return into the first trigger end position T1 as illustrated in FIGS. 7 and 9. Here, the proximal section of the flexible arm 77 will assume its initial configuration and will bend radially outwardly.

Accordingly, the peg 82 of the mechanical switch 81 returns into the first switching state i. Also this return movement of the switch is registered by the processor 42 as a change of the switching state of the switch arrangement 50.

In the present example the second trigger end position T2 is characterized and correlated to a closing or depression of the switch 80 and the second end position of the trigger 70 is characterized by an opening or release of the switch 80. With other examples an inverted configuration can be implemented, wherein the switch 80 is depressed or closed when the trigger reaches the first trigger end position and wherein the switch 80 is opened when the trigger reaches the second end position.

In order to avoid a false logging of dispensing- or injection-related data it is provided that a change of the switching state of the switch arrangement 50 only takes place when directly approaching or reaching one of the first end position P1 and the second end position P2. This way, the switch arrangement 50 and/or the trigger 70 provides a hysteresis function.

In FIG. 13 such a hysteresis function is schematically illustrated. In vertical direction the different switching states of the switch arrangement 50 are illustrated. In horizontal direction the movement of a switch component, such as the peg 82, in transverse, e.g., radial direction r is shown as the trigger 70 or the movable part 16 are subject to a respective movement in longitudinal direction x. As becomes apparent from the illustration of FIG. 13 the switch arrangement is and remains in the first switching state i and may be hence in an open configuration as long as the trigger is either in the first trigger end position T1 and/or is moved from the first trigger end position T1 towards the second trigger end position T2.

It is only upon reaching the second trigger end position T2 that the switch arrangement 50 switches into the second switching state ii. When in the second switching state ii a return motion of the trigger 70 towards the first trigger end position T1 is substantially without effect unless the trigger 70 reaches the first trigger end position T1. It is only upon reaching or approaching the first trigger end position that the switch arrangement 50 swaps into the first switching state i.

This hysteresis function may be either implemented electronically. Here, it is conceivable, that the movement of the peg 82 is gradually monitored, e.g., by the processor 42. It is only upon reaching the respective end position that the processor records or triggers a respective change of the switching state.

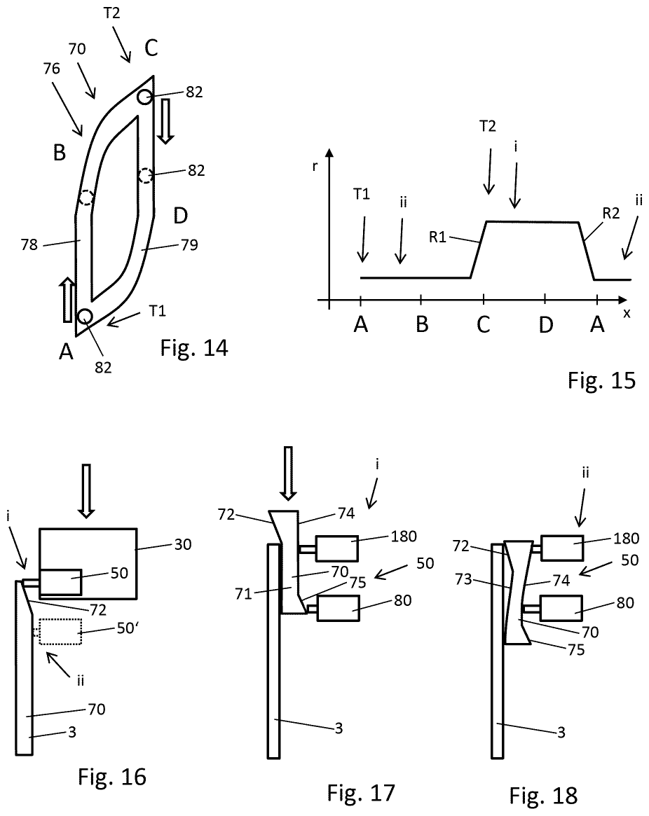

With FIGS. 14 and 15 a mechanical implementation of a hysteresis function is schematically illustrated. Here, the trigger 70 includes a slotted link 76. The slotted link 76 includes a closed loop and further includes a first path 78 and a second path 79. The first and second paths 78, 79 predominantly extend in longitudinal direction x. They do not overlap and are slightly bended in a tangential or circumferential direction with regards to the substantial tubular shape of the housing 3 or of the movable part 16. The peg 82 of the mechanical switch 81 is guided in the slotted link 76. The peg 82 may be in sliding contact with a bottom of the slotted link 76. The bottom of the slotted link may include varying depths in transverse or radial direction as illustrated in FIG. 15.

When the movable part 16 is moved between the first end position P1 and the second end position P2 and hence when the trigger is moved from the first trigger end position T1 towards the second trigger end position T2 the peg 82 slides along the first path 78 or second path 79.

As illustrated in FIG. 14 and when in the first trigger end position T1 the peg 82 is near a position A at the beginning of the first path 78. During a movement of the trigger 70 from the first trigger end position T1 towards the second trigger end position T2 the peg 82 slides along the first path 78, passes beyond a section B and arrives in section C, hence at an opposite longitudinal end of the slotted link 76.

As illustrated in FIG. 15 there is provided a ramp section R1 when approaching the region C and when arriving in the second trigger end position T2. Accordingly, and when approaching or reaching the second trigger end position T2 the peg 82 slides along this ramp section R1 and experiences a respective depression in radial or transverse direction. Thereafter and when the trigger 70 is subject to a movement from the second trigger end position T2 towards the first trigger end position T1 the peg 82 slides along the second path 79 of the slotted link 76. It passes section D of the slotted link 76 and slides along a second ramped section R2 before or upon reaching or approaching the first trigger end position T1. In this way, a switching of the switch arrangement is obtained only in direct vicinity of the longitudinal ends of the slotted link 76, i.e., near the slotted link sections A and C. Moreover, the peg 82 may be biased in transverse or tangential direction so as to have the tendency to be positioned in the first path 78. In this way it can be ensured, that the peg 82, when subject to a movement from position A towards position C, always slides along the first path 78.

In either way and with any conceivable type of hysteresis function implementation it can be provided that a false data logging is prevented, namely, when the movable part is only partially moved from one end position towards the other end position without reaching the another end position. Raising or pushing the button or the movable part 16 only half way or partially between the first and second end positions p1 and p2 will not result in a dose being dispensed and will thus not result in a dose being recorded or logged by the data logging device 30.

The further example of FIG. 16 the data logging device 30 is movably disposed inside the housing 3. Here, a sidewall of the housing 3 may coincide with the trigger 70 or may provide the trigger 70. Here, the housing 3 provides a trigger body 71 with an outside surface 73 and an inside surface 74. At a proximal end or at a proximally facing section the inside surface 74 of the trigger 70 or of the housing 3 includes a beveled section 72. The beveled section 72 is configured to engage with the mechanical switch 81 in a way similar as described above. But here, the data logging device 30 and the switch arrangement 50 are movable in longitudinal direction relative to the housing 3 and hence relative to the trigger 70.

In FIG. 16 the first end position P1 of the movable part 16 is schematically indicated. As the movable part 16 is moved together with the switch arrangement 50 towards the second end position P2, the switch arrangement 50 is moved relative to the trigger 70. The peg 82 of the mechanical switch 81 slides along the beveled section 72 and experiences a respective movement relative to the trigger housing 83. Thus, when arriving in the second end position P2 as indicated by the dotted reproduction of the switch arrangement 50′ in FIG. 16, the switch arrangement 50 has changed its switching state compared to the configuration when the switch arrangement 50 is in the first end position relative to the trigger 70.

In FIGS. 17 and 18 another example of a hysteresis function is illustrated. Here, the switch arrangement 50 includes a first switch 80 and a second switch 180. Here, the switch arrangement 50 is fixed relative to the housing 3 of the injection device 1. The trigger 70 is movable relative to the housing together with the movable part 16. The trigger 70 includes a trigger body 71. The trigger 70 is arranged radially between the first and the second switches 80, 180 and the housing 3. The trigger 70 includes a trigger body 71 featuring an outer surface section 73 facing towards the housing 3. The trigger body 71 further includes an inwardly facing surface section 74 facing towards the first and the second switches 80, 180. The first and second switches 80, 180 are arranged longitudinally offset from each other. In the first end position P1 or in the first trigger end position T1 as illustrated in FIG. 17 a beveled section 75 protruding radially inwardly from the inside surface 74 and provided at the distal end of the trigger body 71 is in engagement with the second switch 180, thereby closing the second switch 180. In this configuration the trigger 70 does not depress the first switch 80. Hence, the first switch 80 is in an open state and the second switch 180 is in a closed state. This configuration is interpreted by the processor that the switch arrangement 50 is in the first switching state i.

As the trigger 70 is subject to a movement towards and into the second trigger end position T2 as illustrated in FIG. 18 a beveled section 72 provided at a proximal longitudinal end of the trigger 70 and facing radially outwardly from the outside facing surface section 73 engages with the sidewall of the housing 3 thereby urging the proximal end of the trigger 70 radially inwardly. Accordingly, the first switch 80 is depressed and closed. Concurrently, the beveled section 75 has moved out of engagement with the second switch 180 and the second switch 180 will be opened. Here, it is particularly intended, that this switch configuration is only and exclusively obtained when the second trigger end position T2 is reached.

In any intermediate position between the first trigger end position T1 and the second trigger end position T2 the switches 80, 180 may be in a common switch state, which may be interpreted as an invalid switching state of the switch arrangement 50 by the processor 42. By implementing a first switch 80 and a second switch 180 a respective hysteresis function as illustrated for instance in FIG. 13 can be obtained. A valid switching state, may require that the first switch 80 and the second switch 180 are in different switching configurations.

With other implementations it is conceivable, that an equal switch configuration of the first switch 80 and of the second switch 180 is regarded as a valid switch configuration and that for any position of the trigger 70 between the first trigger end position T1 and the second trigger end position T2 there is provides a different switch configuration of the first switch 80 and the second switch 180, which is then interpreted as an invalid switching state and is hence disregarded by the processor 42.

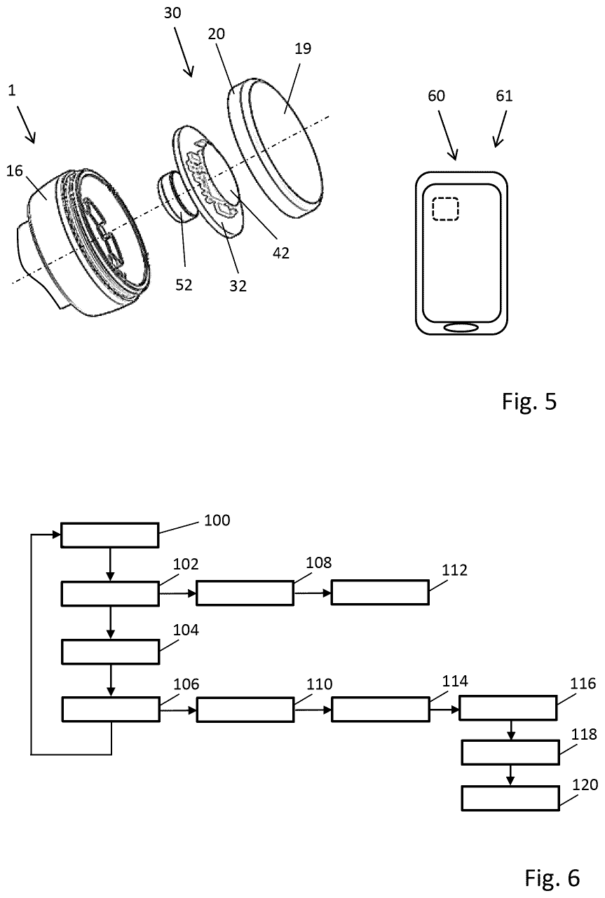

In FIG. 5, a schematic illustration of the data logging device 30 integrated into the injection device 1 is illustrated. Here, the movable part 16, hence in form of a dose button includes a hollow interior. The hollow interior of the movable part 16 allows assembly of the data logging device 30 therein. The data logging device 30 includes a power source 52, e.g., in form of a button battery. The data logging device 30 further includes a printed circuit board 32. On the printed circuit board 32 there is provided at least the processor 42. The processor 42 and the power source 52 may be provided on opposite sides of the printed circuit board 32. The hollow interior of the movable part 16 may be closable by a lid 20. An outside facing end surface of the lid 20 may provide the dispensing surface 19, which is depressible by a user to initiate or to control a dose expelling procedure.

FIG. 5 further illustrates an external electronic device 60. The external electronic device may be implemented as a smartphone 61. The external electronic device 60 may be configured to set up a communication link and hence a data connection 62 with a transceiver 56 of the data logging device 30.

FIG. 4 is a block diagram of an example of the data logging device 30. The data logging device 30 includes processor 42 including one or more processors, such as a microprocessor, a Digital Signal Processor (DSP), Application Specific Integrated Circuit (ASIC), Field Programmable Gate Array (FPGA) or the like, together with a memory 44. The memory 44 may include a program memory and main memory, which can store software for execution by the processor 42 and data generated during use of the data logging device 30 such as a time stamp at which an injection took place.

The data logging device 30 may optionally include a display 48 to visually provide status information of the data logging device 30 and/or information regarding the process of data logging. With some examples the display 48 may be implemented as a touch sensitive display. It may equally serve as an input module.

The processor 42 is operably connected to a switch arrangement 50. The switch arrangement 50 provides electrical signals being indicative of a switching state of the switch arrangement 50. The switching state correlates with the momentary position of the movable part 16 relative to the housing 3. A change in the switching state serves to switch the processor 42 from a sleep mode into a wake-up mode. A change in the switching state further triggers recording of a timestamp in the memory 44.

Additionally, the switch arrangement 50 is connected to the power source 52 of the data logging device 30. In this way, power consumption of the processor 42 can be interrupted or re-established by the switch arrangement 50. With other examples, the processor 42 is permanently connected to the power source 52, while the switch arrangement 50 is located remote from the interface between the processor 42 and the power source 52. Rather the switch arrangement 50 is only logically connected to the processor 42 to switch the processor between a sleep mode and a wake-up mode.

The electronic components of the data logging device 30 are typically mounted on a printed circuit board 32. The printed circuit board 32 together with the switch arrangement 50 provides a dose detection arrangement 40 by way of which setting and/or dispensing of individual doses of the medicament injected by the drug delivery device 1 can be detected and recorded.

The data logging device 30 further includes an interface 54. The interface may provide wired or wireless communication with an external electronic device 60. The interface 54 may include a transceiver 56 providing wireless data transmission and establishing of a wireless data connection 62 with the external electronic device 60. This allows and supports a regular exchange or transmission of data captured or recorded by the data logging device 30 to and/or from the external electronic device 60.

The interface 54 may be a wireless communications interface for communicating with the external device 60, e.g., implemented as a portable electronic device, via a wireless communication link 62 or wireless network, such as Wi-Fi, NFC or Bluetooth®, or an interface for a wired communications link, such as a socket for receiving a Universal Series Bus (USB), mini-USB or micro-USB connector. For this, the interface 54 includes a wireless or wired transceiver 56 configured for transmitting and receiving data.

The data logging device 30 may also include an output module 46. The output module 46 may provide haptic or acoustic feedback, e.g., when a change of the switching state of the switch arrangement 50 has been detected and/or when dose dispensing related information or data is stored in the memory 44, or has been transmitted to the external electronic device 60.

The data logging device 30 may further include a timer 45. The timer may provide a timestamp and time information for storage in the memory 44. Moreover, by way of the timer 45, the processor 42 may autonomously switch into the sleep mode, e.g., after a predefined time interval has lapsed after detection that of a dose dispensing event has terminated.

With some examples the data logging device 30 includes a device housing 31 configured for detachable connection with the drug delivery device 1. With other examples, the data logging device 30 is void of an own housing 31 but is integrated into the housing 3 of the drug delivery device 1.

In FIG. 6 a flowchart of a method of monitoring use of the injection device is illustrated. Here, in a first step 100 the injection device 1 is prepared for a dose injection procedure. In step 100 the user pulls the movable part 16 in proximal direction relative to the housing 3. In the subsequent step 102 the movable part 16 arrives in the first end position P1. In step 102 a switching of the first end position P1 is detected by the switch arrangement 50. Accordingly, the switching state of the switch arrangement 50 is subject to a modification or change in step 108.

Consequently and when arriving in the first end position P1 the switch arrangement 50 may be configured to wake-up the processor 42 of the data logging device. Now and in step 112 a wake-up procedure is conducted by the processor 42. The data logging device 30 is then ready to record a dose dispensing event. In a subsequent step 104 a user pushes the movable part 16 towards the second end position P2. In step 106, the movable part 16 arrives in the second end position P2. This arrival is also registered by the switch arrangement 50 in step 110. The processor 42 is operable to detect a respective change of the switching state of the switch arrangement 50 and correspondingly records the dispensing of a dose in step 114.

In a subsequent step 116 the recorded dose is either transmitted to the external electronic device 60 and/or the detected dose dispensing is stored in a local electronic memory. Thereafter and in step 118 the processor 42 is kept in the wake-up state for a predetermined time interval. After the predetermined time interval has lapsed the processor is set into a sleep mode in step 120.

In this state, energy consumption of the data logging device is reduced to a minimum, thus allowing to prolong the lifetime of the data logging device and use of the electric energy reservoir as provided by the power source 52. With a subsequent dose setting procedure the method will start again with step 100.

### REFERENCE NUMBERS

1 drug delivery device
2 cartridge retaining part
3 housing
4 cartridge
5 piston
6 threaded region
7 internal housing
8 opening
9 thread
10 piston rod
11 pressure foot
12 thread
13 flexible arm
14 drive sleeve
15 threaded part
16 movable part
17 helical thread
18 grip surface
19 dispensing surface
20 lid
22 removable cap
30 data logging device
31 housing
32 printed circuit board
40 dose detection arrangement
42 processor
44 memory
45 timer
46 output
48 display
50 switch arrangement
52 power source
54 interface
56 transceiver
60 external electronic device
61 smartphone
62 data connection
70 trigger
71 trigger body
72 beveled section
73 surface section
74 surface section
75 beveled section
76 slotted link
77 flexible arm
78 first path
79 second path
80 switch
81 mechanical switch
82 peg
83 housing
84 return spring
180 switch

1-15. (canceled)

16. A data logging device for monitoring use of an injection device, the injection device comprising a housing and a movable part, which is movable relative to the housing between a first end position and a second end position along a longitudinal direction, the data logging device comprising:
a trigger connected to or connectable to at least one of the movable part and the housing;
a switch arrangement comprising a first switch operably engaged with the trigger, wherein the trigger is movable relative to the switch arrangement between a first trigger end position and a second trigger end position, the switch arrangement being operable to switch between a first switching state and a second switching state upon movement of the trigger relative to the switch arrangement, wherein the switch arrangement is configured to switch into the first switching state when the trigger approaches or reaches the first trigger end position and wherein the switch arrangement is configured to switch into the second switching state when the trigger approaches or reaches the second trigger end position; and
a processor connected to the switch arrangement and operable to detect a transition of the switch arrangement between the first switching state and the second switching state.

17. The data logging device according to claim 16, wherein the switch arrangement is in the first switching state when the trigger is in the first trigger end position and wherein the switch arrangement remains in the first switching state until the trigger approaches or reaches the second trigger end position.

18. The data logging device according to claim 16, wherein the switch arrangement is in the second switching state when the trigger is in the second trigger end position and wherein the switch arrangement remains in the second switching state until the trigger approaches or reaches the first trigger end position.

19. The data logging device according to claim 16, wherein the processor is operable to selectively enter into one of a sleep mode and a wake-up mode, and wherein the processor is transferable from the sleep mode into the wake-up mode by switching the switch arrangement into the first switch state.

20. The data logging device according to claim 16, wherein the processor is operable to log a dose dispensing event when the switch arrangement is switched from the first switching state into the second switching state.

21. The data logging device according to claim 16, wherein a hysteresis function is provided by at least one of the trigger and the switch arrangement or by the operable engagement of the trigger and the switch arrangement, wherein the hysteresis function is operable to prevent a change of the switching state of the switch arrangement as long as the trigger is located between the first trigger end position and the second trigger end position.

22. The data logging device according to claim 16, wherein the first switch is a mechanical switch comprising a switch housing and a peg protruding from the switch housing, wherein the mechanical switch is movable relative to the switch housing against the action of a return spring.

23. The data logging device according to claim 22, wherein the peg is movable along a transverse direction relative to the switch housing and wherein the trigger, or at least a portion thereof, is movable, pivotable, or bendable in the transverse direction when the trigger moves between the first trigger end position and the second trigger end position.

24. The data logging device according to claim 22, wherein the trigger comprises a trigger body movable relative to the housing along the longitudinal direction, wherein the trigger body comprises a beveled section which, when engaged with the housing, is operable to induce at least one of a movement, a pivoting, or a bending of the trigger, or at least a portion thereof, in the transverse direction.

25. The data logging device according to claim 16, wherein the switch arrangement comprises a first switch and a second switch, wherein each of the first switch and the second switch is connected to the processor and wherein each of the first switch and the second switch is operably engaged with the trigger, wherein the first switch is operable to detect the trigger approaching or arriving at the first trigger end position, and wherein the second switch is operable to detect the trigger approaching or arriving at the second trigger end position.

26. The data logging device according to claim 25, wherein the first switch or the second switch comprises one of an electromechanical switch, a magnetic switch, an optical sensor, or an acoustic sensor.

27. The data logging device according to claim 16, further comprising a logging device housing configured for attachment to the housing of the injection device, and wherein the trigger is configured for attachment to the movable part of the injection device.

28. An injection device for injecting of a dose of a medicament into biological tissue, the injection device comprising:
a housing to accommodate a cartridge filled with an injectable medicament and sealed towards a proximal direction by a piston movable relative to a barrel of the cartridge,
a piston rod to operably engage with the piston to expel a dose of the medicament from the cartridge,
a movable part movable relative to the housing between a first end position and a second end position along a longitudinal direction, and
a data logging device comprising:
a trigger connected to or connectable to at least one of the movable part and the housing;
a switch arrangement comprising a first switch operably engaged with the trigger, wherein the trigger is movable relative to the switch arrangement between a first trigger end position and a second trigger end position, the switch arrangement being operable to switch between a first switching state and a second switching state upon movement of the trigger relative to the switch arrangement, wherein the switch arrangement is configured to switch into the first switching state when the trigger approaches or reaches the first trigger end position and wherein the switch arrangement is configured to switch into the second switching state when the trigger approaches or reaches the second trigger end position; and
a processor connected to the switch arrangement and operable to detect a transition of the switch arrangement between the first switching state and the second switching state.

29. The injection device according to claim 28, wherein the data logging device is arranged inside at least one of the housing and the movable part, and wherein the trigger is connected to the movable part.

30. The injection device according to claim 28, wherein the cartridge filled with the injectable medicament is arranged inside the housing.

31. The injection device according to claim 28, wherein the trigger is integrated into the movable part and wherein the trigger comprises a resilient arm to slidably engage with a sidewall of the housing when the movable part is moved between the first end position and the second end position.

32. The injection device according to claim 28, wherein the injection device is transferable from an idle state into a cocked state by moving the movable part from the second end position towards and into the first end position.

33. The injection device according to claim 28, wherein a fixed dose of the medicament is injectable by moving the movable part from the first end position towards and into the second end position.

34. The injection device according to claim 28, wherein the piston rod is movable in a longitudinal distal direction by and through moving the movable part from the first end position towards and into the second end position.

35. The injection device according to claim 28, wherein a longitudinal displacement of the movable part relative to the housing from the first end position towards and into the second end position is proportionally transmitted into a respective longitudinally directed advancing displacement of the piston rod for injecting of the dose of the medicament.
